(19) United States
(12) Patent Application Publication
(10) Pub. No.: US 2005/0128434 A1
(43) Pub. Date: Jun. 16, 2005

(54) Computerized virtual reflex perimetry

(76) Inventors: Tsontcho Ianchulev, Los Angeles, CA (US); Peter Pham, Long Beach, CA (US); Vladimir Makarov, Pasadena, CA (US)

Correspondence Address: Peter Pham, 1175 East Second Street #17, Long Beach, CA, 90802, US

(21) Appl. No.: 10/946,949

(22) Filed: Sep. 22, 2004

Related U.S. Application Data
(60) Provisional application No. 60/506,845, filed on Sep. 30, 2003.

(51) Int. Cl. A61B 3/02; A61B 3/00; A61B 13/00

(52) U.S. Cl. 351/239; 600/558; 351/246

(57) ABSTRACT

An automated unaided virtual perimetry system and underlying method for relative-threshold contrast sensitivity interrogation of the visual field via algorithmic presentation of visual stimuli on a screen and subsequent analysis of test response data for the detection of pathophysiologic scotomas as well response time registration and analysis for visual motor reflex testing. Launch of system via access from a remote server or direct installation of systems technology allows seamless capability at the local computer system to instruct, administer, and analyze output of stimuli and input of responses of test subjects or subjects independent of external input or control. Specific algorithmic test environment is designed to achieve comparable clinical efficacy and diagnostic utility to office-based perimetry system.

### CROSS-REFERENCE TO RELATED APPLICATION

This application claims priority to U.S. provisional application Ser. No. 60/506,845 filed Sep. 30^{th}, 2003.

### TECHNICAL FIELD

This invention relates to the methods and systems for automated visual field (perimetry) testing.

### BACKGROUND OF PRESENT INVENTION

Recent progress in medical science has enabled significant extension to average life expectancy with a marked improvement in the activity and lifestyle of the elderly. Octogenarians drive and perform more and more complex and involved tasks of daily life. These higher demands on performance necessitate the development of a system for monitoring and benchmarking of visual and reflex abilities. Without an adequate visual capacity, such as an intact and physiologic visual field, or an unimpaired visual motor reflex, the ability to drive, fly, or perform other ordinary activities could be greatly diminished. As a result, the safety and wellbeing of the public can be affected.

Visual-Motor reflex capacity is tantamount for the qualified driver. In most states, applicants are required to demonstrate adequate visual function, such as a satisfactory visual acuity and/or visual field, before a license is authorized. In order to qualify for a driver's license in the United States, one must not only pass the visual acuity test but also the visual field test. To obtain the initial license and in many cases to maintain a license in later years, a driving test with direct supervision is needed in which judgment as well the visual motor reflex of the driver is assessed. While judgment cannot be measured objectively, visual motor reflex is easily measured.

Visual motor reflex can be defined as the interval of time needed to initiate a motor activity, be it pressing on the brakes or swinging a bat, in response to a visual stimulus. Events that require immediate action while driving a car or flying a plane often are often not those that are directly in front of view but those that occur in the periphery. The ability to assess visual motor response in the peripheral field is an important component that until now has not been well addressed because there has been no method to date to measure it without the use of complicated and dedicated equipment.

A number of methods and systems have been developed to assist in the enhancement and measurement of motor skills. In U.S. Pat. No. 5,803,745 to Kozak, et al. a system is described to help develop motor coordination. An invention in U.S. Pat. No. 6,066,105 to Guillen, describes an apparatus to measure the motor response time consisting of special anunciators and sensors to monitor a subject's response to stimulation. There are also a variety of patents that use game to improve motor skills (U.S. Pat. Nos. 6,722,888, 5,846,086). No invention has addressed visual motor reflex as it relates to visual field testing. Described in this patent is a method and system to assess both the visual field and visual motor reflex at any point in the visual field. This unique ability allows for the screening of diseases such as glaucoma that affect the visual field as well as insights into the response time within the visual field.

The subject of visual field is complex and expansive; the following is a brief synopsis of it as it relates to our invention. Vision consists of more than just visual acuity or the ability to distinguish or resolve two separate points in space. The commonly used Snellen chart is widely used to test for visual acuity. However visual acuity alone will not confer enough functional vision for basic tasks such as reading, walking, or driving if the visual field, or the area in space that is visible, is constricted to a pinpoint, as commonly occurs in advanced glaucoma. Since ancient times, it's been known that the visual field is an integral part of vision.

Defects in the visual field are known as scotomas and represent dysfunctional areas in the peripheral vision, which have decreased sensitivity to visual stimulation. For each eye normal visual field implies a standard range that subtends approximately 120 degrees vertically and 150 degrees horizontally. Because the visual fields of each eye overlap, a severe loss of visual field function in one eye may not be noticed until it is well advanced and begins to affect central visual field and acuity. Early detection through with visual field screening is essential to avoid irreversible damage. Pathophysiologic scotomas or “blind areas” characterize a number of diseases, most importantly glaucoma, optic neuritis, cerebrovascular accidents and others.

Perhaps the most common and challenging disease associated with visual field defects is glaucoma. Glaucoma is a blinding disease that affects the function of the optic nerve. It often leads asymptomatic impairment of the visual field caused by underlying loss of optic nerve tissue. Early in the course of glaucoma, the peripheral visual field becomes less sensitive to light and the affected areas turn into scotomas. In the very advanced stages of the disease, the visual field can be so damaged that only 5-10 degrees of visual field are left intact. Since the central vision (i.e. fixation) is not affected, the visual acuity may be normal (20/20). The insidious nature of glaucoma causes more than half of those affect to be unaware of their disease in the United States (H. A. Quigley and S. Vitale, Models of open-angle glaucoma prevalence and incidence in the United States. Invest Ophthalmol Vis Sci 38 (1997), pp. 83-91.) (Sommer A, Tielsch J M, Katz J, Quigley H A, Gottsch J D, Javitt J & Singh K (1991): Relationship between intraocular pressure and primary open-angle glaucoma among white and black Americans: the Baltimore Eye Survey. Arch Ophthalmol 109: 1090-1098._) and over 75% in Japan (Y. Shiose, Y. Kitazawa, S. Tsukahara et al., Epidemiology of glaucoma in Japan—a nationwide glaucoma survey. Jpn J Ophthalmol 35 (1991), pp. 133-155.), even in advanced cases. There is currently no reliable, effective method to screen for glaucoma in the general population. Current methods are ineffectual when applied on a large scale.

Perimetry is an objective method to test the integrity of the peripheral vision and its clinical application dates back more than a century. The first attempt at perimetry is attributed to von Graefe who developed the first method of testing with a simple grid chart mounted on a wall (Graefe's Archiv 2:258, 1856). Since then perimetry has evolved into technologically advanced automated systems that enable fast, standardized, operator-independent testing for sensitive and specific detection of visual field defects. Each perimetry method has its advantages and disadvantages. A simple wall-mounted perimeter using a wand and circular target points may be appropriate for an experienced clinician in an office setting where only a gross estimation of the visual field is needed, but it would be too crude and impractical to serve as a screening method. More complex perimetric devices can map the visual field in great detail but would require expensive dedicated equipment and specially trained technician.

Logistically, all perimetry systems employ the same fundamental principles but differ mainly in their algorithms or methods used to carry out the testing. Despite the differences there are several cardinal principles, which, like inviolable laws of physics, must be followed in any perimetry evaluation—from the simplest to the most advanced. The principles are:
1. Positioning—Positioning refers to the location of the tested eye relative to testing surface. The distance the tested eye away from the testing surface of a given dimension determines the breadth of visual field that can be tested and is easily calculated with simple geometry. The degree of visual field tested is therefore simply the inverse tangent of the ratio of testing surface dimension to the distance of the tested eye away the testing surface. In the early tangent screen systems, the test subject is positioned at a preset distance, and that position is maintained by continuous observation from the operator, who also employs intermittent testing at the physiologic blind spot to verify proper positioning. The physiologic blind spot, first described by Mariotte in 1668, is an small area 15 degrees away from fixation where the optic nerve enters the eye and there is no responsive nerve tissue to transmit visual signal (Edme Mariotte: Nouvelle Découvverte touchant la veüe. A Paris chez Frederic Leonard, MDCLXVIII. [Blind Spot]). Hence any stimulus falling on the blind will not be perceived and positive responses at this position imply improper positioning or fixation. In the case of modern office perimetry systems like the Humphrey and the Octopus analyzers, the eye position is maintained by a mechanical chin rest and head band that be adjusted until the proper eye position is produced. Failure to maintain proper produces unreliable test results and renders perimetry testing meaningless.
2. Fixation—Fixation refers to the maintenance of the direction of gaze. Visual field examination cannot be performed without reliable fixation. In order to test the peripheral vision, stimuli must be presented away from the point of fixation. Since there is a natural tendency to “foveate” or fixate on incident stimulation by “searching” for targets in the field of view, it is important to design a system that abolishes this reflex mechanism and keeps the eye fixated on a target while testing the response to stimuli in the periphery. No matter how good the fixation system may be for any perimetry device, it can never be better than the ability of the test subject to fixate.
3. Presentation of stimuli at different points in the visual field to test the ability of the subject to recognize that stimuli. This step is repeated until the desired breadth and detail of visual field is tested. The stimulus or background on which the stimulus rests may vary in size, space, contrast, time interval, or any combination thereof such that almost anything from a hand or lasers can act as the stimulus target.
4. Reliability Assurance—For a test to be standardized and reproducible, subjective variability such as loss of fixation, fatigability, and response consistency need to be monitored and controlled. Ideally, subjects would always look at the fixation target and respond only to stimuli when they are presented. In reality, this optimal scenario is rarely attainable. In order to gauge the reliability of the test, perimetry systems rely on either direct observation by the operator or indirect reliability parameters such as fixation losses, false positives and false negative or both. With direct observation, the test subject is directly observed by the operator to ensure that positioning is preserved, fixation is maintained, and instructions are followed. Indirect reliability assessment involves stimulus presentation at the blind spot to check for fixation loss or improper positioning. When the tested eye is correctly positioned and foveating, any stimulus at the blind spot will not be perceived and there should be no positive responses from the test subject. False positives can be tested by generating extraneous noises or presenting stimuli that are too dim to be detectable. False negative can be tested by presenting stimuli that are brighter than prior stimuli at same locations that were previously registered to be detected. Failure to detect the more intense stimuli indicates a false negative response.
5. Analysis—Analysis involves the compilation of all the data points and information gathered during testing. The depth of analysis depends on how the test was designed and administered. Devices such as the Humphrey Field Analyzer allows for statistical analysis while others such as a tangent screen give an accurate plot of the location of scotomas and an approximate depth of defects at those locations. When used for screening purposes a simple analysis of whether pathophysiologic scotomas are present or not is sufficient. However, in patients already diagnosed with glaucoma, it is important to know not only the location of the scotomas but its depth or severity at those locations in order to assess the effectiveness of treatment.

One of the most common and well-known visual field testing device is the Humphrey Visual Field Analyzer, whose methodology is based on U.S. Pat. No. 4,349,250 to Gelius. Using a statistical modeling testing procedure, the Humphrey Field Analyzer is able to produce consistent, quantitative results in a reproducible environment, allowing for comparison among tests taken by a test subject at different times. This allows for the monitoring of field defect progression over time to aid in treatment and intervention. This is also used to document visual field defects in subjects suspected to having such defects based on clinical data. Another testing method similar to the Humphrey Field Analyzer is the Octopus device whose methodology is based on U.S. Pat. No. 4,334,738 to Seckinger. However both these methods are ill suited for deployment to screen for visual field defect. It would be prohibitively expensive and impractical to use either of these dedicated devices for such a task because of the minimum requirement to test hundreds to millions of subjects in an efficient and cost-effective manner.

Several other methods have been proposed and patented to facilitate the visual field testing. They vary from manual, handheld campimetric techniques to more intricate electronic means. U.S. Pat. No. 5,886,770 to Damato describes handheld campimetry device with a rotating knob to present stimuli that appear at different test areas on the fixed grid. Either a ruler or the physiologic blind spot can be used to locate the appropriate distance of the test subject away from the testing apparatus. This method requires someone other than the test subject to administer the test (B. E. Damato, J. Chyla, E. McClure, J. L. Jay and D. Allan, A hand-held OKP chart for the screening of glaucoma: preliminary evaluation. Eye 4 (1990), pp. 632-637.).

A more involved method is described in U.S. Pat. No. 6,033,076 to Braeuning, et al. consisting of a neural network controlling the input and output of data in real time based on a test subject's response at a remote site where the visual field test is administered. The main requirement for this method is the need for a continuous interface between the remote and local site exchange data and commands in a rapid, uninterruptible manner such that the output control from the remote site is dependent on the output data response from the local site where the test subject is located. This sequence of dependent controlled input-output flow is repeated until the entire visual field is mapped.

Another method of visual field testing using computer technology is described in U.S. Pat. No. 5,565,949 to Kasha. A laptop computer is used as the platform for testing in this case. A moving fixation point is also used to increase in the viewable degrees of visual field as well as a strategy to improve compliance during testing. The fixation point is allowed to move within a “bounding box” within which the stimuli location calculated in relations to the position of the fixation spot. A very similar method is described in U.S. Pat. No. 4,995,717 to Damato whereby a computer monitor rather than a laptop monitor is used as the platform. The stimuli are arranged radially relative to a moving fixation point. In this method the both the moving fixation point and stimuli arranged radially to it are moved as a whole unit.

In U.S. Pat. No. 5,946,075 to Horn, color contrast symbols on a colored background act as stimuli for visual field testing. Calibration of the testing environment involves the use of devices to measure and adjust ambient light, employing such tools as light meters or luminescent light cards. In a later related patent also ascribed to Horn, U.S. Pat. No. 6,260,970, a moving fixation target is used to maintain fixation such that a test subject must continually place a cursor on the fixation target for further testing to proceed. Failure to align the cursor, which is moved with the aid of a mouse input, causes suspension of the test and prompts instructions to be given for the test subject to align the cursor with the moving fixation target again.

In the above descriptions of several different visual field testing methods and patents, there is one or more differing themes to each patent but each rely on the previously 5 outlined steps for testing the visual field of a test subject.

### OBJECT AND SUMMARY OF PRESENT INVENTION

We wish to present a novel system and method employing all the fundamental principles of visual field testing without the need for dedicated, bulky, and costly equipment as found in the Humphrey Visual Field Analyzer or Octopus System or any other type of dedicated testing apparatus. In addition our method involves analysis of the response time to gauge the test subject's visual motor reflex, which in addition to visual field analysis gives valuable insights into a subjects ability to drive or perform other tasks that require some degree of visual motor reflex. Although dedicated visual field systems produce consistent results that can be compared over time, they are impractical to use on a large scale, such as for population based screening, where it is estimated that over 50% of those with glaucoma are unaware of their disease.

It is well known that the best method to screen for glaucoma is with visual field rather than with intraocular pressure measurements or optic nerve examination (J. M. Tielsch, J. Katz, K. Singh et al., A population-based evaluation of glaucoma screening: the Baltimore Eye Survey. Am J Epidemiol 134 (1991), pp. 1102-1110.), (P. Mitchell, W. Smith, K. Attebo and P. R. Healey, Prevalence of open-angle glaucoma in Australia. The Blue Mountains Eye Study. Ophthalmology 103 (1996), pp. 1661-1669.) (Delgado M F, Nguyen N T, Cox T A, Singh K, Lee D A, Dueker D K, Fechtner R D, Juzych M S, Lin S C, Netland P A, Pastor S A, Schuman J S, Samples J R; American Academy of Ophthalmology. Ophthalmic Technology Assessment Committee 2001-2002 Glaucoma Panel.). It is also known that many if not most patients with definitive glaucoma have introcular pressures in the “normal” range (Bengtsson B (1989): Characteristics of manifest glaucoma at early stages. Graefe's Arch Clin Exp Ophthalmol 227: 241-243.), (Klein B E K, Klein R, Sponsel W E, Franke T, Cantor L B, Martone J & Menange M J (1992): Prevalence of glaucoma. The Beaver Dam Eye Study. Ophthalmology 99: 1499-1504.) The assessment of optic disc appearance requires an experienced clinician in order to obtain any degree of clinical accuracy but even with the best-trained eyes, there is substantial overlap between normal and glaucomatous disc appearance (A. L. Coleman, A. Sommer, C. Enger et al., Interobserver and intraobserver variability in the detection of glaucomatous progression of the optic disc. J Glaucoma 5 (1996), pp. 384-389.), (The sensitivity and specificity of direct ophthalmoscopic optic disc assessment in screening for glaucoma: a multivariate analysis.Graefes Arch Clin Exp Ophthalmol. 2000 December; 238(12):949-55.) (Tielsch J M, Katz J, Singh K, Quigley H A, Gottsch J D, Javitt J, Sommer A., A population-based evaluation of glaucoma screening: the Baltimore Eye Survey. Am J Epidemiol. 1991 Nov. 15; 134(10):1102-10.).

Our system does not rely on a continuous, simultaneous feedback neural network loop interface to administer the test. The problems inherent in establishing and maintaining such a complex system to cohesively connect both the remote and local site in the real world would be difficult to accomplish in a practical fashion. Our visual field system also would not rely moving fixation target or variations in color contrast recognition. The use of a moving fixation point to increase degrees of viewable visual field on a small monitor or compliance during testing would be impractical for the targeted audience of older individuals who are most are risk for visual field defects from glaucoma where the amount of eye-hand coordination required is often unachievable. The implementation of color contrast symbols with fine-tuning through the use of light meters poses both logistical and technical problems. Many if not most households do not possess luminescent materials or light meters and the ability of different monitors to display a uniform set brightness and contrast is difficult to achieve with so many different manufacturers and standards in the marketplace.

Our invention is distinct in its systematic virtual design. It uses internet technology for in-demand delivery but is fully functional without a direct link to the internet since it can be also be installed from a disc or other recordable media and be completely functional without external electronic loop. It uses simple, easily achievable specification parameters that are readily standardized among numerous types of monitor display devices to achieve clinically relevant results that have been validated in a clinical trial (reference). This system is accessible to almost anyone with internet access or the most basic of computer system. This confers the ability not only to perform visual field testing on a mass screening basis but also the option of using it as an office based system. In a related implementation of our system, visual motor reflex testing can also be performed. Clinical trial with our systems demonstrated results of our visual field testing system correlated highly with those of the Humphrey Field Analyzer. Furthermore in the population studied in the clinical trial, over 40% were computer illiterate but were able to comprehend and complete the test without difficulty, highlighting the user-friendly nature of our system.

### BRIEF DESCRIPTION OF DRAWINGS

FIG. 1:

Flowchart of the basic steps involved in performing our visual field testing system.

FIG. 2:

Angular relations of visual field area, fixation marker, and blind spot marker.

FIG. 3:

Diagram of novel positioning method using a discontinuous vertical line centered on the blind spot marker.

FIG. 4:

A static schematic of appearance of test screen during testing.

FIG. 5:

Diagram of visual motor reflex algorith.

### DESCRIPTION OF THE PREFERRED EMBODIMENT

What follows describes the preferred embodiment of our system but does not in any way limit the myriad of ways our system and its components can be modified, upgraded or altered without violating its form or function. It would be impossible to fully and completely convey all the variations in a few pages of text and diagrams but anyone skilled in the arts would readily recognized the possibilities inherent in our system.

Our system relies on proprietary, clinically validated systems integration among various components consisting of two primary parts:
1) Internet-based software that is deployable via the internet or on electronic media such as compact discs.
2) Computer system with a CPU, storage device, viewing surface, browser interface, and input device such as a keyboard, mouse, or trigger switch.

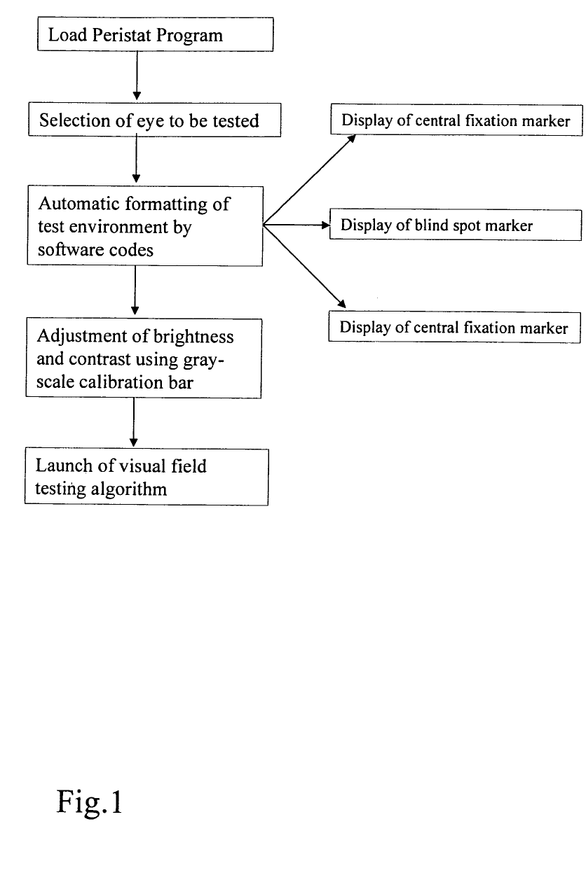

In detail, FIG. 1 outlines the basic steps involved during visual field testing with our system. The software is easily downloading via the internet from a server or can be directly transferred to the computer system on an electronic media. Once the software is launched and the test subject or subjects are presented with a brief instructional session to the test subject or subjects prior to testing on the display screen that is connected to the computer system. This display screen can be of any light emitting or light reflecting surface such as a cathode ray tube, LCD monitor, or even a wall or screen (light reflecting surface) onto which a video projection system illuminates the area. The option to display on a large screen the size of a movie theater confers for a unique feature to this system whereby a large number of subjects can be tested simultaneously simply by having multiple subject view the same large screen. Each subject would have an input device to respond the stimuli presented on the screen. Because the screen size is large, there would be an insignificant difference in the angular viewing angle when multiple subjects are clustered centrally with respect to the screen. The software automatically adjusts all the parameters of the test such as the location of the blind spot, fixation spot, the stimuli location without any input from the test subject. Only the contrast level of the monitor needs adjustment by the test subject by using a relative gray-scale grid presented on the display screen. The use of a gray scale rather than color variation facilitates greater standardization among the numerous types of monitor available. A gray scale is characterized by equal inputs of red, green, and blue. The only variance is luminance. Each primary color as well as hue, saturation, and luminance is adjusted on a scale of 0-255 in customary 8-bit system. At the extremes of “black” and “white” the luminance as well as the 3 primary colors varies from 0 to 255, respectively. By eliminating individual variations in each of the 3 primary colors that would be needed to produce various shades of orange or brown for instance, there is less propensity for marked, individual variation among monitors. Using a relative rather than absolute scale further reduces variation in the testing parameters. Besides the technical aspects of why a relative gray value scale is optimal for visual field testing, perhaps the most important is that it has been clinically validated with a patient trial comparing it to the gold standard for automated visual field testing, the Humphrey Visual Field Analyzer. The following steps proceed in logical order from position the test subject at the appropriate distance away from the viewing screen to initiation of the actual visual field screening test when the stimuli are repeated presented until the visual field is mapped and analyzed.

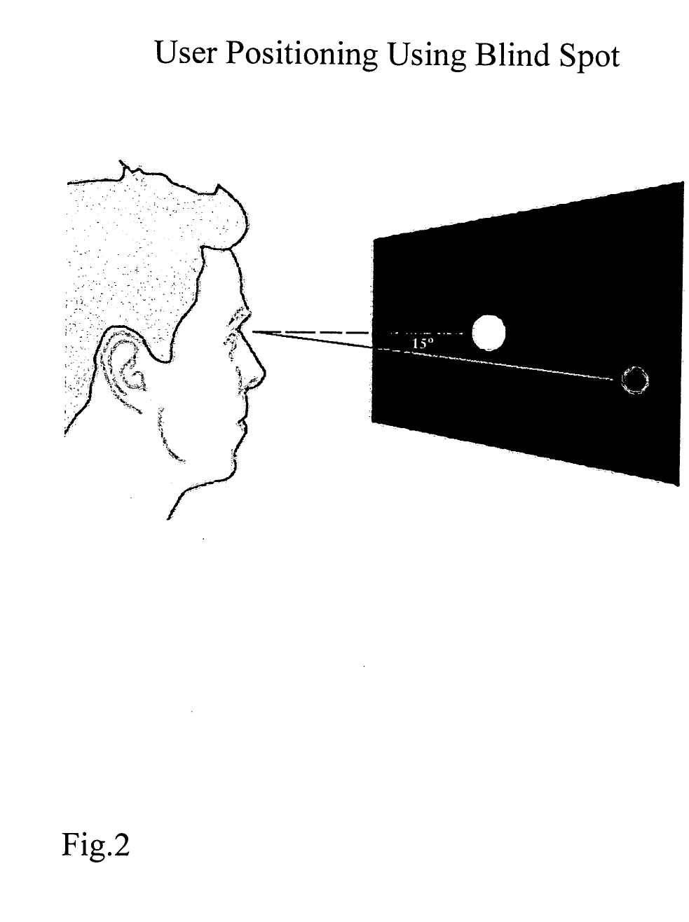

FIG. 2.

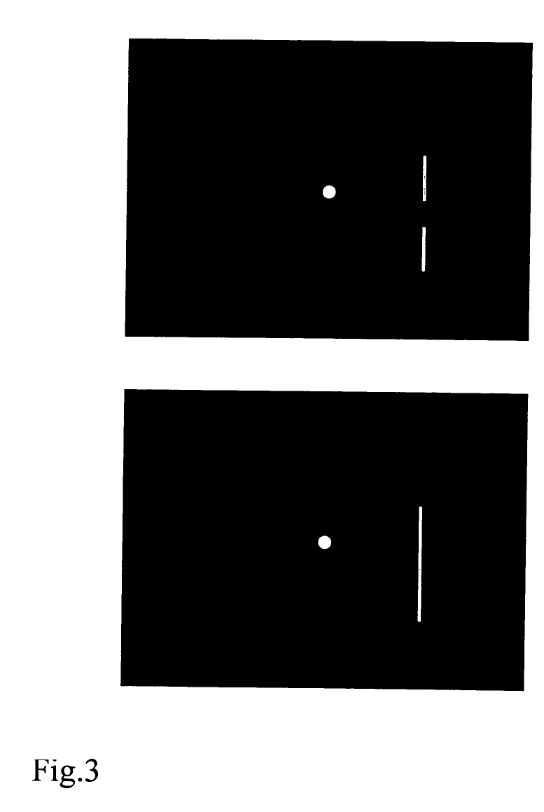

Proper position of a test subject from the testing screen is crucial for the reliability and performance of any perimetry system. In our system, positioning can either be done with a preset ruler or marker for that specific screen size and desired breadth of visual field examination. This distance value is easily calculated by those skilled in the arts as half the viewable horizontal screen size divided by the tangent of 25°, given the fact that the preferred embodiment our system tests 25° of visual field away from fixation horizontally. Another method of positioning, shown in FIG. 2, involves the use of the blind spot, which is the preferred embodiment of our system since this technique requires no additional accessories. It is known that the blind spot 2 (right eye), is situated 15° temporal to fixation (1) (Edme Mariotte: Nouvelle Découvverte touchant la veüe. A Paris chez Frederic Leonard, MDCLXVIII.), and the size of the blind spot measures 40 horizontally and 6° vertically about its center. The blind spot exists because physiologically, the optic nerve lies in this area. Objects or images falling within this area will not be detected. However there is no noticeable gap or hole in the visual field around this area because of a phenomenon known as “fill-in” effect in which the brain “fills” in the missing area without conscious input or knowledge. If a test subject, with one eye covered and the other focusing on the fixation point, slowly moves in from a position approximately 1.5 times the horizontal dimension of the screen size towards the screen, there will be a point where the entire blind spot mark “disappears” from view. At this point, the test subject is correctly positioned. Although not evident from the diagram, the blind spot mark flashes continuously on the screen. This enables to test subject to automatically adjust positioning if the blind spot mark appears again. The blind spot will also reappear if the test subject does not focus on the fixation spot. We have also developed another novel method of positioning, as shown in FIG. 3, that not only relies on the principle of blind spot location, which is well known in the arts, but also takes advantage of the “fill-in” effect described earlier. In this method, as the test subject slowly moves in from a distance approximately 1.5 times the horizontal dimension of the screen size towards the screen while focusing on the fixation spot (1), the discontinuous line (3) suddenly becomes a continuous one as the central circle (3) corresponding to the blind spot disappears from view. Using this positioning method, there are now two cues to prompt the test subject to adjust positioning if necessary, the re-emergence of the blind spot mark or the reappearance of a discontinuous line. Moreover with this novel method, there is now a constant, visible marker delineating to the test subject that there is proper positioning. The embodiment of how our system performs the tasks of visual field testing will become clear from the following description of FIG. 3.

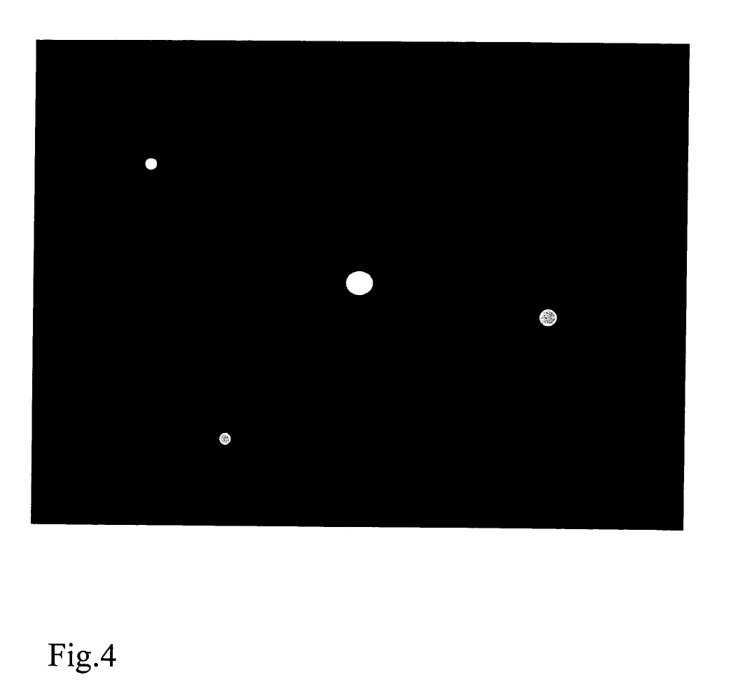

FIG. 4 embodies the form and function of our system but in no way captures the entire essences of it as there can be numerous iteration of our system without altering the foundation of our system. As shown, there is a central fixation spot (1) with blind spot markers (2). Not shown is the fact that the blind spot marker continuously flashes throughout the test to aid in the proper positioning of the test subject as described previously. Also shown are different luminance levels the stimuli (4, 5, 6) that are intermittently presented throughout the test. By varying the luminance of the stimuli, the depth of visual field defects can be assessed. The preferred embodiment of our system uses three difference luminance levels for screening to detect between mild, moderate, and severe levels of visual field defect depending on which of the luminance level was missed during testing. Throughout the test, there are also reliability parameters consisting of false positive, false negative, and fixation loss. These parameters are crucial to determining how accurately and reliably the test subject performed on the test. Although not directly shown in FIG. 3 but well known in the arts, our system registers a false positive whenever a test subject responds with a click when there was no stimulus displayed on the screen and conversely, a false negative occurs whenever a test subject fails to respond the highest luminance level at the same location where a lower luminance level was detected earlier. Fixation loss is calculated by displaying the highest luminance stimuli (7) in the area of the blind spot. Recognition of this stimulus is indicative of improper fixation on the fixation spot. It should be evident that our system is able to carry out more detailed, quantitative examination of the visual field than with just a few luminance levels and it is also obvious that the degrees of visual capable of being test is not limited to 20-25° but is in fact much greater or smaller than this simply by adjusting the positions of the different markers and stimuli presented.

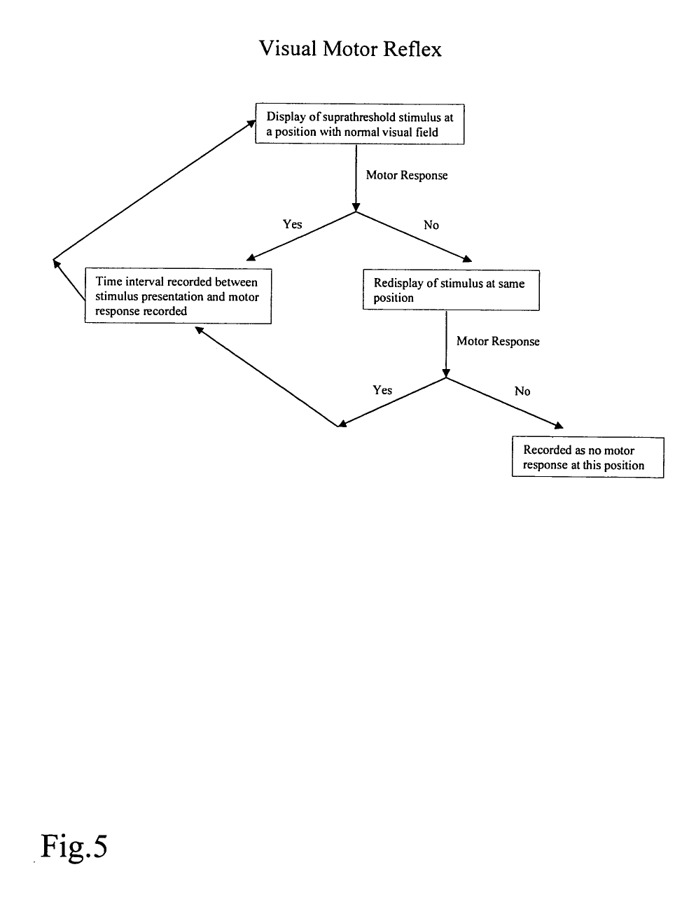

FIG. 5 details the algorithmic method used to assess visual motor reflex at any point in the visual field. The measurement of visual motor reflex is best done a brief rest following visual field examination or alternatively in can also be done during the visual field test. By utilizing the reliability parameters previously described for visual field screening, the reliability of the visual motor reflex examination can also be gauged. The algorithm entails testing points the visual field that have previously been shown on visual field examination to be normal in each of the four quadrants of the visual filed. The number of points tested in each quadrant can be varied according to the required degree of sophistication required. The response time is calculated as the interval between stimuli presentation and stimuli acknowledgement. The stimulus can be visual or auditory depending on the capability of the local system. The results from visual motor reflex testing can then be displayed in a tabular or graphical format. From the previous detailed description of the embodiment of our system,

1. An integrated system for visual field testing consisting of customized software codes, computer system comprising of computer processing unit, storage unit, display screen, and input device to qualitatively and/or quantitatively assess the visual field of a subject or subjects without requiring outside personnel support.

2. Means for accessing or installing systems technology in claim 1 using the internet or any electronic data media. Computer processing unit to execute said software codes on internet or in said electronic data media.

3. Means for displaying output data of said computer processing unit consisting of a light reflecting and/or light emitting surface according to claim 1

4. Means for recording responses consisting of input device such as a mouse, keypad, or foot switch to said output data in claim 1 for said subject or subjects.

5. Method for formatting test environment for visual field testing using said software codes consisting of following

6. automatic internal calibration of screen resolution is performed by said software codes.

7. gray-scale calibration bar generated by said software codes on said screen for user to adjust said screen brightness and resolution. Said screen calibration is correctly adjusted when the lowest gray scale calibration bar matches the background of said screen.

8. Display of dynamic central fixation marker on said screen. Said dynamic central fixation marker may consist of a numeric counter that changes numerals at a specified time interval according to claim 5. Said changes in numerals are timed at specified interval by said software codes. Said dynamic fixation marker may be of any geometric shape that dynamic changes over time to help maintain the interest of the user.

9. Presentation of blind spot marker at 15 degrees temporal to the central fixation spot according to claim 7. Said blind spot marker may be any geometric shape continuously flashes to help user monitor eye position. Alternatively, said blind spot marker may be a discontinuous line.

10. Presentation of selection buttons for right or left eye.

11. Method according to claim 5 whereby user selects the desired eye to be tested by clicking on appropriate right or left eye button with said input device. After selection, said user covers non-tested eye and fixates on central fixation marker with the eye to be tested. Said users then positions head by placing head near the center of said display surface. Positioning is performed by moving head towards display surface until blind spot marker is no longer visible or alternatively if said discontinuous line is used, until a “continuous” line is detected.

12. Method according to claim 5 whereby said user presses on input device to engage said software to interrogate the desired visual field.

13. Method to analyze visual field of said user using said software code initiates a series of algorithmic steps that are independent of external control or input culminating in the complete analysis of the desired visual field breadth. Said algorithmic steps include

14. Display of stimulus marker at predetermined points for predetermined time interval on said display surface. Preferred embodiment of said stimulus marker is a gray-scale circular stimulus but may be of any shape, size, or color. Preferred embodiment of time interval is 0.3 seconds but stimulus frequency may be varied to test different subsets of retinal ganglion cells. Said time interval between stimulus presentation may be adjusted depending on response rate of said user.

15. Retesting of points that were not detected by user with a stimulus marker of higher intensity until user response is detected or until highest intensity stimulus marker is displayed.

16. Assessment of fixation loss by presentation of stimulus marker in the blind spot. Detection of stimulus marker at blind spot connotes fixation loss.

17. Assessment of false positive response by varying the timing of stimulus presentation with said fixation counter. Response by user when no stimulus is presented connotes a false positive response.

18. Assessment of false negative response by displaying a stimulus marker at higher intensity in a location where a previous stimulus marker was detected at a lower intensity. Failure to respond at the higher intensity stimulus marker connotes a false negative.

19. Method according to claim 5 of analyzing and displaying said subject's responses.

20. Method for testing said user reflex response at any point in visual field using said test environment according to claim 5 consisting of.

21. Presentation of stimulus marker at highest intensity level for 0.25 second at desired visual field location where prior visual field analysis had indicated normal visual field function.

22. Time registration of user response via input device after cessation of stimulus marker stimulation

23. Repetition of prior steps throughout visual field until desired breadth of reflex perimetry is obtained

24. Said input device my be computer mouse or foot pedal

25. Analysis and display of response time from said user
